(19) United States
(12) Patent Application Publication
(10) Pub. No.: US 2008/0260695 A1
(43) Pub. Date: Oct. 23, 2008

(54) Composition for prevention and/or treatment of cancer

(75) Inventor: Kung-Ming Lu, Taipei City (TW)

Correspondence Address: BIRCH STEWART KOLASCH & BIRCH, PO BOX 747, FALLS CHURCH, VA, 22040-0747, US

(73) Assignee: MICROBIO COMPANY LTD.

(21) Appl. No.: 11/826,482

(22) Filed: Jul. 16, 2007

(30) Foreign Application Priority Data
Apr. 20, 2007 (TW) 096114009

(51) Int. Cl. A61K 35/74; A61K 36/48; A61K 36/82; A61K 36/87; A61P 35/00

(52) U.S. Cl. 424/93.4; 424/729; 424/757; 424/766

(57) ABSTRACT

The invention relates to a composition for use in the prevention and treatment of cancer. The composition includes fermented soy extract, oligomeric proanthocyanidin, epigallocatiechin gallate, spirulina, curcumin and Antrodia camphorata. The composition of the invention effectively prevents the growth of cancer cells, especially colorectal cancer, ovarian cancer, breast cancer, cervical cancer, and liver cancer.

### BACKGROUND OF THE INVENTION

1. Field of the Invention

The present invention relates to pharmaceutical composition, and in particular relates to anti-cancer composition.

2. Description of the Related Art

Cancer is estimated to be the second most prevalent disease in populations worldwide. Nearly all cancers of the organs are incurable. Presently, the treatment of the cancer includes surgery, radiation therapy, immunotherapy, chemotherapy, and alcohol injection among others. Most anti-tumor drugs have a higher toxicity to rapidly proliferating cancer cells (such as Leukemia, or lymphoma) than to slowly proliferating cancer cells (such as liver cancer, or lung cancer). Many anti-cancer drugs additionally exhibit poor selectivity to cancer cells, resulting in undesirable side effects. A feasible alternative is to explore active ingredients in Chinese herbal medicines as adjuvants or primary therapies for cancer treatment.

Cancer chemoprevention involves use of natural or pharmaceutical agents to prevent, slow or halt the process of carcinogenesis. These agents inhibit the development of invasive cancer either by blocking the DNA damage that initiates carcinogenesis or by diverting the progression to a benign outcome, such as apoptosis or differentiation of these precancerous cells. Chemopreventive agents may be defined as substances that reduce the synthesis of carcinogens in the body, chemicals that enhance their detoxification by Phase I or Phase II enzymes, antioxidants that scavenge free radicals, and chemicals that trap ultimate carcinogens preventing their interaction with DNA. Because chemopreventive agents must be administered over a long period of time in order to determine their effectiveness in a human host, it is of paramount importance that the chemopreventive agents be non-toxic and relatively free from side effects to determine the effectiveness thereof. For many candidate agents, mechanisms of action can be well characterized using human or other mammalian cells propagated in vitro, whereas potential toxic effects can often be predicted by studying administration to animals in vivo. The agents must further be taken orally, in pill, food or beverage form and easily modifiable in order to increase convenience and the likelihood that human subjects comply with daily consumption requirements.

Dietary epidemiological studies of cancer development have generated new clues about micronutrients and other dietary components to act as efficacious cancer preventive agents. For example, intake of soybeans and soy-based products is associated with a lower risk of several types of cancers including breast, prostate and colon cancer. Experiments in various animal models also suggested that soy consumption could decrease tumor number, incidence, latency, multiplicity and metastasis. Thus, a highly safe and composition for prevention and treatment of cancer is desirable.

### BRIEF SUMMARY OF INVENTION

The invention provides a composition for preventing and/or treating cancer. The composition comprises fermented soy extract, oligomeric proanthocyanidin, epigallocatiechin gallate, spirulina, curcumin, and Antrodia camphorata.

A detailed description is given in the following embodiments with reference to the accompanying drawings.

### BRIEF DESCRIPTION OF DRAWINGS

The present invention can be more fully understood by reading the subsequent detailed description and examples with references made to the accompanying drawings, wherein:

FIG. 1 shows that the composition of the invention did not damage F344 rats;

FIG. 2a-2h show images of crypt multiplicity of aberrant crypt foci (ACF) in rat colon when subject to methylene blue stain (ACF assay) under light microscope;

FIG. 3a-3d show images of crypt multiplicity of ACF in rat colon during the hematoxylin-eosin stain under light microscope;

FIG. 4 shows that the composition of the invention did not damage F344 rats;

FIG. 5a shows images of the normal colorectal tissues under light microscope;

FIG. 5b-5c shows images of DMH(dimethylhydrazine)-induced ACF under light microscope;

FIG. 6a shows images of normal tissue under light microscope;

FIG. 6b shows images of the adenocarcinoma in the initial stages of tumor under light microscope, and

FIG. 6c shows images of the abnormal tissue in the later stage of tumor under light microscope.

### DETAILED DESCRIPTION OF INVENTION

The following description is of the best-contemplated mode of carrying out the invention. This description is made for the purpose of illustrating the general principles of the invention and should not be taken in a limiting sense. The scope of the invention is best determined by reference to the appended claims.

The invention provides a composition for prevention and treatment of cancer. The composition comprises fermented soy extract, oligomeric proanthocyanidin, epigallocatiechin gallate, spirulina, curcumin, and Antrodia camphorata. A weight ratio of soy extract, oligomeric proanthocyanidin, epigallocatiechin gallate, spirulina, curcumin, and Antrodia camphorata is about 12-30:1:4:2:1:1, more preferably, 12-20:1:4:2:1:1, most preferably, 12:1:4:2:1:1. The composition of the invention adhering to the described conditions shows high efficacy in the prevention and treatment of cancer. In addition, the weight ratio may be adjusted to meet different conditions.

The fermented soy extract is by fermentation of an aqueous soy extract with at least one lactic bacteria, such as Lacctobacillus spp., one yeast, such as Saccharomyces spp., Saccharmoyces cerevisiae, etc. or both and is subsequently sterilized, filtered, and concentrated. The details of fermented soy extract were discussed in detail in U.S. Pat. No. 6,855,350, and the fermented soy extract also can be a commercial product sold by Microbio Biotechnology Company. Additionally, the oligomeric proanthocyanidin may be a grape seed proanthocyanidin extract, and the epigallocatechin gallate may be a green tea epigallocatechin gallate (EGCG).

The composition of the invention is administered orally, or by injection. The invention can be administrated alone, or in combination with a pharmaceutically acceptable carrier, dilutant, excipient or combinations thereof. The pharmaceutically acceptable carrier can include solvents, dispersion media, coatings, antibacterial and antifungal agents, isotonic and absorption delaying agents, and the like. The pharmaceutically acceptable dilute can include such as bacteriostatic water for injection (BWFI), phosphate-buffered saline, Ringer's solution and dextrose solution. The pharmaceutical excipient can include calcium carbonate, sodium carbonate, lactose, calcium phosphate, sodium phosphate, maize starch, alginic acid, starch, gelatin or acacia.

The composition can repress the DMH to reduce the number of ACF, and the repression rate exceeds about 40%. The composition also can reduce the number of the ACF, such as reduce 30% of the number. In addition, the composition of the invention can repress ACF of different size, preferably, large size ACF (above 7 crypts).

Another embodiment of the composition of the invention may reduce the number of tumors in excess of about 30% or more. Specifically, small size tumors are reduced by about 40% or more.

The composition of the invention prevents and/or treats cancer so that the composition can be administrated to cancer patient, chemotherapy patient, and high risk group of cancer, etc. In addition, the composition is very safe and does not cause biological damage so that the composition also can be worked as a food supplement.

The composition is effective for prevention and treatment of cancers including, breast, prostate, blood, colorectal, uterine, ovarian, endometrial, cervical, testicular, malignant lymphoma, rhabdomyosarcoma, neuroblastoma, pancreatic, lung, brain, skin, gastric, liver, kidney, and nasopharyngeal cancers. Preferably the composition is used in the prevention and treatment of colorectal, lung, ovarian, breast, cervical, and liver cancers.

### EXAMPLE

### Example 1

### Manufacture of the Composition of the Invention

The composition of the invention comprises fermented soy extract (Microbio Biotechnology®), oligomeric proanthocyanidin, epigallocatiechin gallate, spirulina, curcumin, and Antrodia camphorate, and the weight of each component is listed in Table 1.

TABLE 1
the weight of each component

| Component | Weight (mg) |
|---|---|
| Fermented soy extract (Microbio | 600 |
| Biotechnology ®) | |
| Grape seed (95% oligomeric | 50 |
| proanthocyanidin) | |
| Green tea (50% epigallocatiechin gallate) | 200 |
| Spirulina | 100 |
| Curcuma longa (95% curcumin) | 50 |
| Antrodia camphorata | 50 |
| Total | 1050 |

### Example 2

### Composition of the Invention Prevents Colorectal Cancer

The six components (fermented soy extract (Microbio Biotechnology®), oligomeric proanthocyanidin, epigallocatiechin gallate, spirulina, curcumin, and Antrodia camphorata) of Example 1 were classified to seven groups; the content of each group is listed in Table 2. Each group was respectively diluted 300×, 600×, 1200×, and 2400× with McCoy's 5a medium, and then colorectal cancer cell (HT-29, 80000 cell/well) line was treated with the diluted group A to group G. After 24 hours, the relative survival rates of the colorectal cancer cell line were detected by MTT assay. In a control group, the relative survival rate was 100%. Referring to Table 3, groups D-G (two or more components) had a better repression effect than groups A-C (single component), wherein the effect of group D was best. In addition, the fermented soy extract, curcumin, and epigallocatiechin gallate (group E) had an obvious killed effect and activity relationship. The results are listed in Table 3.

TABLE 2
Components of each group

| Group | Component |
|---|---|
| A | fermented soy extract (Microbio Biotechnology ®) |
| B | epigallocatiechin gallate |
| C | curcumin |
| D | epigallocatiechin gallate + curcumin |
| E | fermented soy extract (Microbio Biotechnology ®) + |
| | epigallocatiechin gallate + curcumin |
| F | oligomeric proanthocyanidin + epigallocatiechin gallate + |
| | spirulina +curcumin + Antrodia camphorata |
| G | Composition of the invention |
| Control | 90% McCoy's 5a medium + Fetal Bovine Serum (FBS) |
| Negative | doxorubicin |
| control | |

TABLE 3
Relative survival rate of colorectal cancer cell line

| | Relatively survival rate (%) | | | |
|---|---|---|---|---|
| | | | | 300 × |
| Group | 2400 × dilution | 1200 × dilution | 600 × dilution | dilution |
| A | 101 | 84 | 78 | 77 |
| B | 81 | 66 | 50 | 23 |
| C | 71 | 45 | 31 | 18 |
| D | 66 | 42 | 24 | 13 |
| E | 59 | 28 | 13 | 11 |
| F | 46 | 27 | 17 | 12 |
| G | 42 | 20 | 14 | 12 |

### Example 3

### Composition of the Invention Prevents Cervical Cancer, Ovarian Cancer, Liver Cancer, and Breast Cancer

The same procedure carried out in Example 3 was repeated with the exception that the colorectal cancer was changed to cervical cancer cell line (HeLa), ovarian cancer cell line (CHO-K1), liver cancer cell line (HepG2), and breast cancer cell line (MCF-7), and groups A to G were changed to groups 1 to 5. The contents of groups 1 to 5 are listed in Table 4. Referring to Tables 5 to 8, the composition of the invention represses the growth of cervical cancer cell line, ovarian cancer cell line, liver cancer cell line, and breast cancer cell line, and the repressed effect of group 2 (the inventive composition) is better than other groups.

TABLE 4
Components of each group

| Group | Component |
|---|---|
| 1 | oligomeric proanthocyanidin + epigallocatiechin gallate + |
| | spirulina +curcumin + Antrodia camphorata |
| 2 | Composition of the invention |
| 3 | oligomeric proanthocyanidin + spirulina + curcumin + |
| | Antrodia camphorata |
| 4 | oligomeric proanthocyanidin + epigallocatiechin gallate + |
| | spirulina + curcumin |
| 5 | oligomeric proanthocyanidin + epigallocatiechin gallate + |
| | spirulina + Antrodia camphorata |
| Control | HepG2, HeLa, and MCF7: 90% MEM medium + 10% FBS |
| | CHO-K1: 90% Ham's F12K medium + 10% FBS |

TABLE 5
Relative survival rate of cervical cancer cell line

| Group | Relatively survival rate (concentration of each group) | | | |
|---|---|---|---|---|
| 1 | 43.8 (0.5 mg/ml) | 20.6 (1 mg/ml) | 19.9 (2 mg/ml) | 19.2 (3 mg/ml) |
| 2 | 33.4 (0.5 mg/ml) | 24.4 (1 mg/ml) | 19.1 (2 mg/ml) | 19.0 (3 mg/ml) |
| 3 | 72.9 (0.5 mg/ml) | 58.0 (1 mg/ml) | 54.2 (2 mg/ml) | 54.0 (3 mg/ml) |
| 4 | 100.0 (0.5 mg/ml) | 80.6 (1 mg/ml) | 61.2 (2 mg/ml) | 60.2 (3 mg/ml) |
| 5 | 61.5 (3 mg/ml) | 31.7 (4 mg/ml) | 20.9 (5 mg/ml) | 20.0 (6 mg/ml) |

TABLE 6
Relative survival rate of ovarian cancer cell line

| Group | Relatively survival rate (concentration of each group) | | | |
|---|---|---|---|---|
| 1 | 55.1 (0.5 mg/ml) | 49.3 (1 mg/ml) | 31.6 (2 mg/ml) | 24.2 (3 mg/ml) |
| 2 | 57.3 (0.5 mg/ml) | 45.7 (1 mg/ml) | 32.5 (2 mg/ml) | 22.5 (3 mg/ml) |
| 3 | 100.0 (0.5 mg/ml) | 85.7 (1 mg/ml) | 85.3 (2 mg/ml) | 78.0 (3 mg/ml) |
| 4 | 100.0 (0.5 mg/ml) | 100.0 (1 mg/ml) | 100.0 (2 mg/ml) | 100.0 (3 mg/ml) |
| 5 | 100.0 (3 mg/ml) | 87.5 (4 mg/ml) | 80.1 (5 mg/ml) | 73.0 (6 mg/ml) |

TABLE 7
Relative survival rate of liver cancer cell line

| Group | Relatively survival rate (concentration of each group) | | | |
|---|---|---|---|---|
| 1 | 33.3 (0.5 mg/ml) | 32.5 (1 mg/ml) | 25.2 (2 mg/ml) | 25.0 (3 mg/ml) |
| 2 | 36.9 (0.5 mg/ml) | 33.0 (1 mg/ml) | 23.9 (2 mg/ml) | 23.4 (3 mg/ml) |
| 3 | 96.1 (0.5 mg/ml) | 81.3 (1 mg/ml) | 61.5 (2 mg/ml) | 52.3 (3 mg/ml) |
| 4 | 100.0 (0.5 mg/ml) | 100.0 (1 mg/ml) | 100.0 (2 mg/ml) | 100.0 (3 mg/ml) |
| 5 | 100.0 (3 mg/ml) | 82.7 (4 mg/ml) | 81.1 (5 mg/ml) | 62.6 (6 mg/ml) |

TABLE 8
Relative survival rate of breast cancer cell line

| Group | Relatively survival rate (concentration of each group) | | | |
|---|---|---|---|---|
| 1 | 92.2 (0.5 mg/ml) | 84.2 (1 mg/ml) | 66.3 (2 mg/ml) | 58.3 (3 mg/ml) |
| 2 | 20.8 (0.5 mg/ml) | 13.6 (1 mg/ml) | 11.8 (2 mg/ml) | 10.8 (3 mg/ml) |
| 3 | 63.8 (0.5 mg/ml) | 57.2 (1 mg/ml) | 56.9 (2 mg/ml) | 43.8 (3 mg/ml) |
| 4 | 100.0 (0.5 mg/ml) | 100.0 (1 mg/ml) | 87.4 (2 mg/ml) | 75.7 (3 mg/ml) |
| 5 | 100.0 (3 mg/ml) | 99.5 (4 mg/ml) | 90.1 (5 mg/ml) | 88.5 (6 mg/ml) |

### Example 4

### Composition of the Invention Reduces the Incidence Rate of ACF

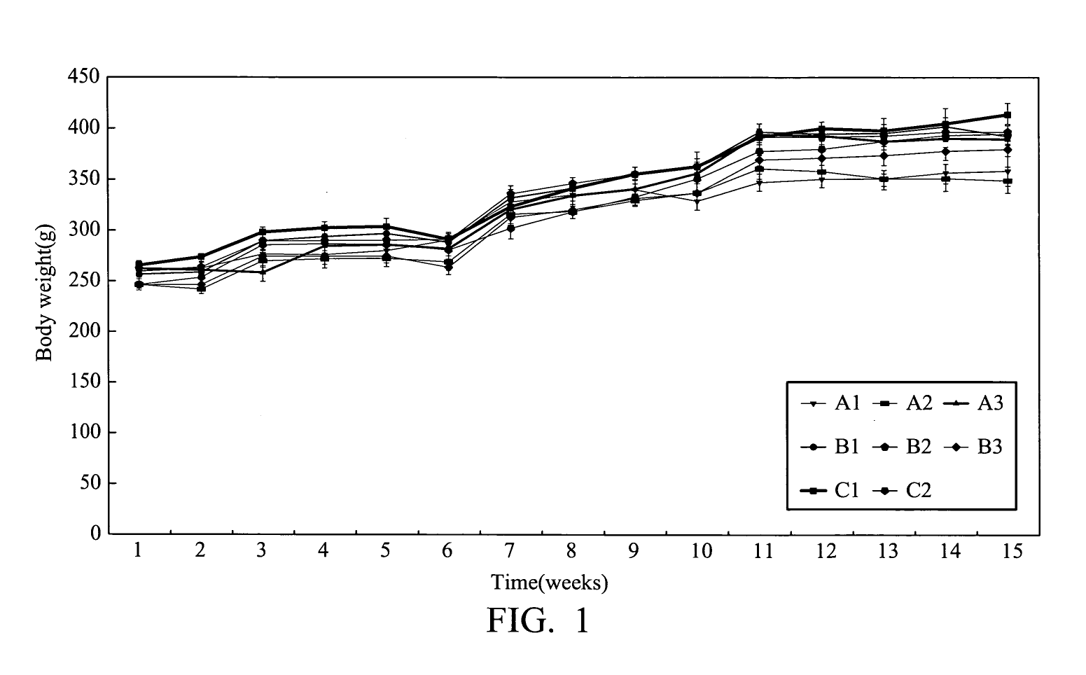

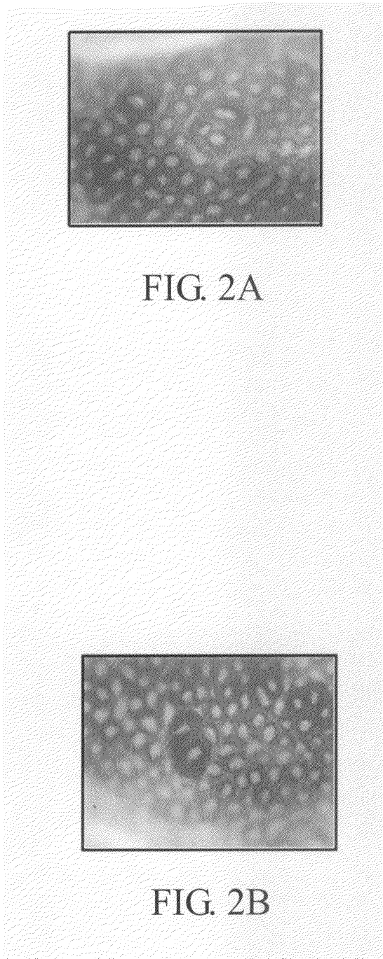

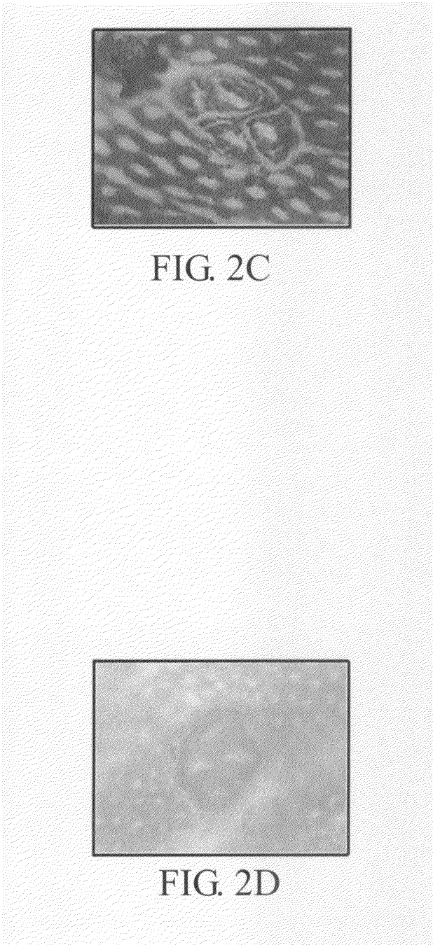

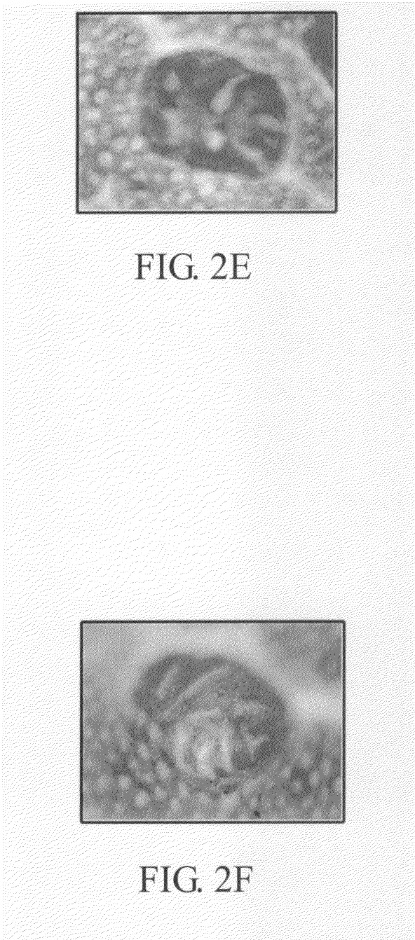

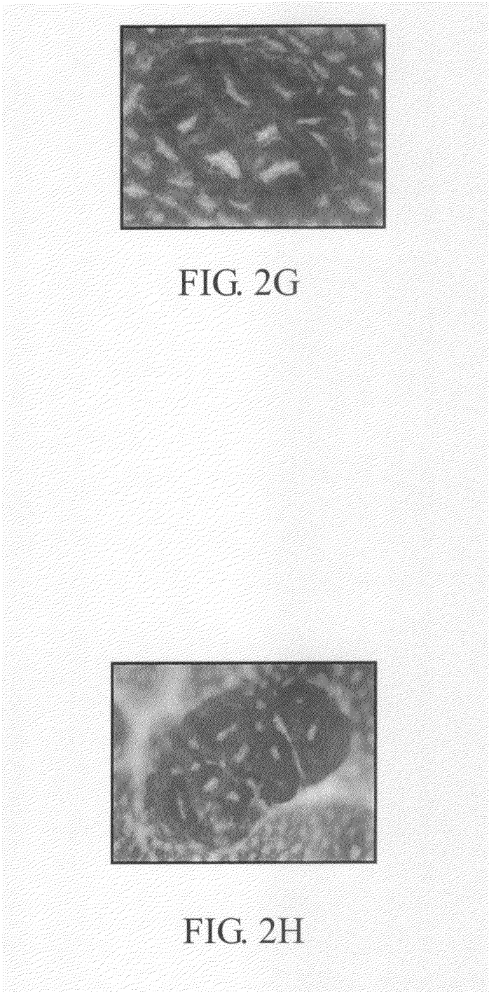

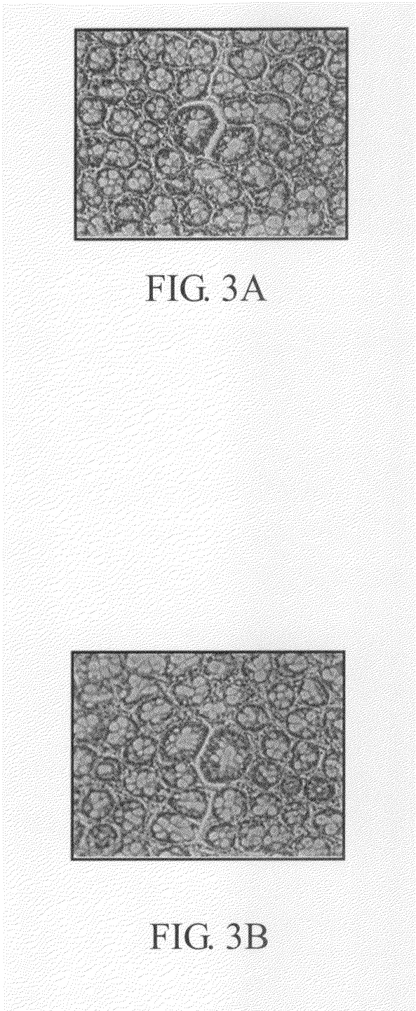

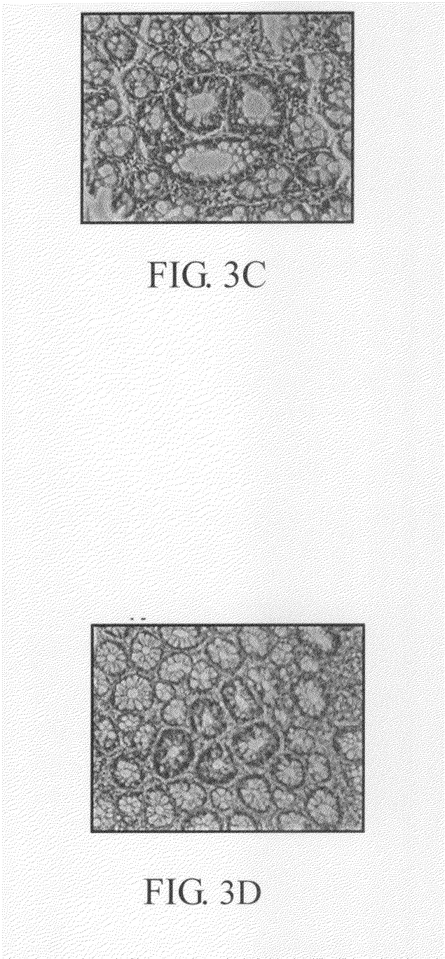

Male F344 rats were classified into eight groups, 8 rats in each group, and the conditions of the experiment are listed in Table 9. The dose of 20 mg/kg, pH 6.7 of DHM solution was injected into each rat every week for 4 weeks (the second week to the fifth week) by intraperitoneal injection (Takahashi, 1993; Taniyama, 2000; Futakuchi, 2002; Ononse, 2003; Hirose, 2002). After 15 weeks, F344 rats were starved for 1 day, and then sacrificed under ether anesthesia. The sacrificed rats were analyzed by ACF assay (See, e.g., Brid, 1998). Referring to FIG. 1, the body weight of each F344 rats did not change significantly during the experiment. Therefore, the composition of the invention did not cause the damages of F344 rats. FIG. 2a-2h show images of crypts of ACF during the methylene blue stain under light microscope (FIG. 2a shows 1-2 crypts, FIG. 2b shows 2 crypts, FIG. 2c shows 4 crypts, FIG. 2d shows 4 crypts, FIG. 2e shows 6 crypts, FIG. 2f shows 8 crypts, FIG. 2g shows 9 crypts, FIG. 2h shows 17 crypts). FIG. 3a-3d show images of crypts of ACF during the hematoxylin-eosin stain (FIG. 3a-3b show 2 crypts, FIG. 3c shows 4 crypts, FIG. 3d shows 5 crypts). The composition of the invention reduced the number of ACF and crypts, wherein repression rate of ACF exceeded about 40-50%, and the repression rate of crypts exceeded 30%. The results were statistically significant.

In addition, ACF could be classified into 3 types, type 1 (1˜3 crypts), type 2 (4˜6 crypts), and type 3 (≧7 crypts) based on number of the crypt. The composition of the invention repressed ACF of different size, preferably, large size ACF (≧7 crypts).

TABLE 9
Experiment conditions

| | | Amount of | |
|---|---|---|---|
| | | treatment | Injection |
| Group | Treatment | (mg/day) | (i.p.) |
| C1 | — | — | DHM |
| C2 | — | — | Normal sailne |
| A1 | fermented soy extract (Microbio | 3.6 | DHM |
| | Biotechnology ®) | | |
| A2 | fermented soy extract (Microbio | 7.1 | DHM |
| | Biotechnology ®) | | |
| A3 | fermented soy extract (Microbio | 14.3 | DHM |
| | Biotechnology ®) | | |
| B1 | Composition of the invention | 17.9 | DHM |
| B2 | Composition of the invention | 35.7 | DHM |
| B3 | Composition of the invention | 71.4 | DHM |

DHM: dimethylhydrazine

TABLE 10
Suppression of ACF

| | No. of ACFs/colon^{a} | Mean no. of crypts/ACF |
|---|---|---|
| Group | (% of inhibition)^{b} | (% of inhibition)^{b} |
| C1 | 148 ± 28.6 (0%) | 4.3 ± 0.0.3 (0%) |
| C2 | 0 | 0 |
| A1 | 95.5 ± 18.2 (35.5%) | 3.0 ± 0.1 (30.2%) |
| A2 | 93.5 ± 27.6 (36.9%) | 3.2 ± 0.3 (25.6%) |
| A3 | 97.8 ± 19.8 (34.0%) | 3.7 ± 0.2 (14.0%) |
| B1 | 82.9 ± 18.7 (44.0%) | 2.9 ± 0.2 (32.6%) |
| B2 | 72.6 ± 16.5 (51.0%) | 3.6 ± 0.4 (16.3%) |
| B3 | 88.0 ± 15.3 (40.6%) | 3.7 ± 0.2 (14.0%) |

^{a}The total number of ACF in all regions examined.
^{b}The decreased number of ACF of experimental groups compared with C1 group.

TABLE 11
Suppression of different ACF

| | No. of ACFs/colon (% of Inhibition)^{a} | | |
|---|---|---|---|
| Group | 1~3 crypts | 4~6 crypts | ≧7 crypts |
| C1 | 64.0 ± 9.3 (0%) | 60.1 ± 14.9 (0%) | 24.0 ± 7.8 (0%) |
| C2 | 0 | 0 | 0 |
| A1 | 59.9 ± 12.8 (6.4%) | 26.5 ± 5.7 (60.0%) | 3.8 ± 1.0 (84.2%) |
| A2 | 55.6 ± 19.1 (13.1%) | 27.6 ± 8.9 (54.1%) | 6.3 ± 3.5 (73.8%) |
| A3 | 46.0 ± 11.4 (28.1%) | 39.8 ± 8.8 (33.8%) | 7.6 ± 2.8 (68.3%) |
| B1 | 48.0 ± 6.1 (25.0%) | 23.1 ± 5.2 (61.6%) | 4.4 ± 1.9 (81.7%) |
| B2 | 37.6 ± 8.2 (41.2%) | 22.6 ± 6.3 (62.4%) | 7.4 ± 2.7 (69.2%) |
| B3 | 44.8 ± 6.8 (30.0%) | 36.5 ± 7.6 (39.3%) | 6.8 ± 2.0 (71.7%) |

^{a}The decreased number of ACF of experimental groups compared with C1 group.

### Example 5

### Composition of the Invention Reduces the Rate of Incidence of Colorectal Cancer

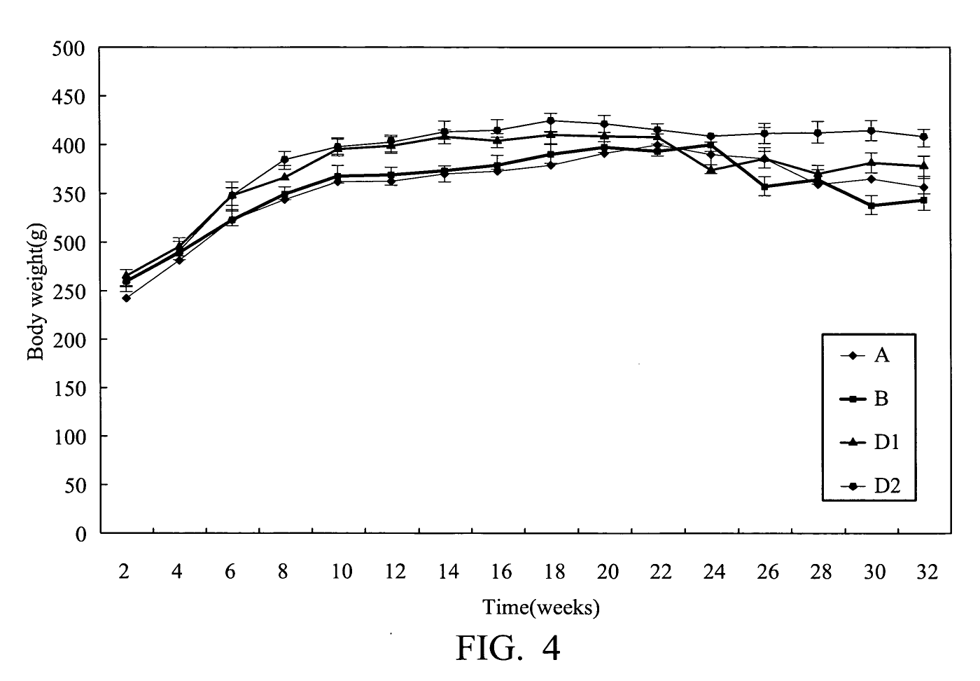

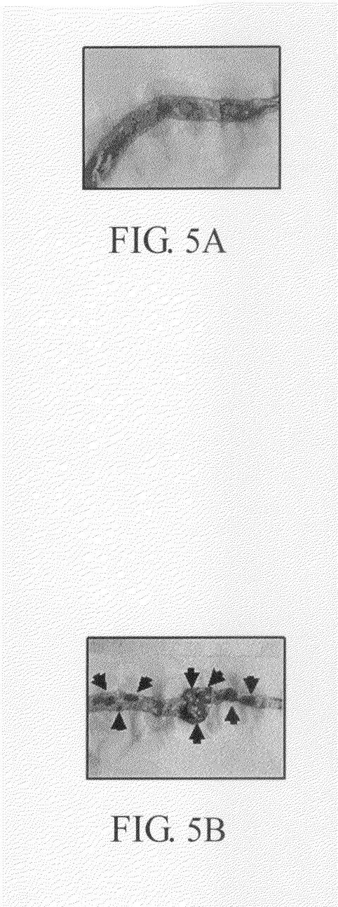

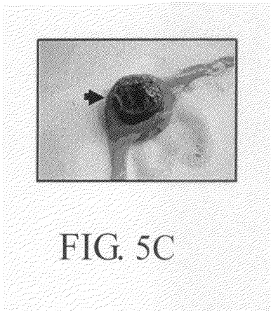

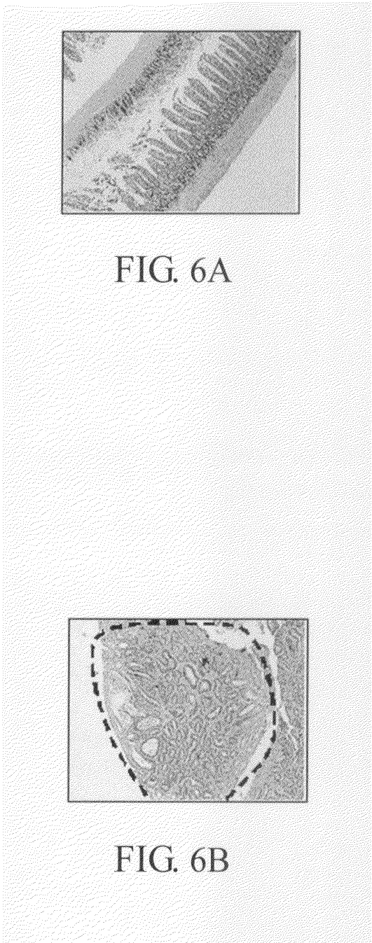

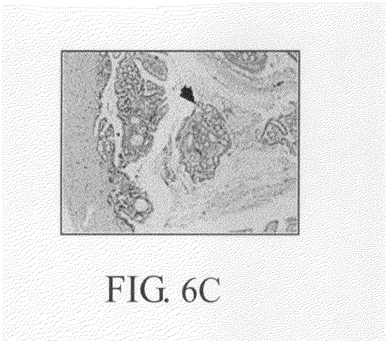

The same procedure carried out in Example 4 was repeated with the exception that the duration of the experiment was changed to 42 hours. The conditions of the experiment are listed in Table 9. FIG. 4 shows that the body weight of each F344 rat did not change significantly during the experiment. Thus, the composition of the invention did not cause damage to F344 rats. FIG. 5a shows normal colorectal tissues, and FIG. 5b-5c shows DMH-induced ACF. FIG. 6a shows the normal tissue, FIG. 6b shows the adenocarcinoma in the initial stages of tumor, and FIG. 6c shows the abnormal tissue in the later stage of tumor.

The composition of the invention reduced the number of tumors in excess of about 38%, the result is listed in Table 13. Specifically, the small size tumor was reduced in excess of about 49.7%, the result is listed in Table 14.

TABLE 12
Experimental conditions

| | | Amount of | |
|---|---|---|---|
| | | treatment | Injection |
| Group | Treatment | (mg/day) | (i.p.) |
| D1 | — | — | DHM |
| D2 | — | — | Normal sailne |
| A | fermented soy extract (Microbio | 3.6 | DHM |
| | Biotechnology ®) | | |
| B | Composition of the invention | 17.9 | DHM |

DHM: dimethylhydrazine

TABLE 13
Tumor suppression rate

| Group | No. of tumors/colon (% of Inhibition) |
|---|---|
| D1 | 16 ± 2.07 (0%) |
| D2 | 0 |
| A | 14.67 ± 2.09 (2.7%) |
| B | 9.85 ± 1.39 (38.4%) |

TABLE 14
Tumor suppression rate in different tumor volume

| | No. of tumors/colon (% of Inhibition)^{b} | | |
|---|---|---|---|
| Group | V <100 mm³ | 100 < V < 500 mm³ | V >500 mm³ |
| D1 | 12.23 ± 1.46 (0%) | 3.08 ± 0.65 (0%) | 0.69 ± 0.19 (0%) |
| D2 | 0 | 0 | 0 |
| A1 | 10.47 ± 1.86 (14.4%) | 3.07 ± 0.67 (NS) | 3.8 ± 1.0 (NS) |
| A2 | 55.6 ± 19.1 (49.7%) | 27.6 ± 8.9 (NS) | 6.3 ± 3.5 (NS) |

V: Tumor volume
^{b}The decreased number of tumors of experimental groups compared with D1 group
NS No statistical significance.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

1. A composition for treating cancer, comprising an effective amount of fermented soy extract, oligomeric proanthocyanidin, epigallocatechin gallate, spirulina, curcumin, and Antrodia camphorata, wherein a weight ratio of the fermented soy extract, oligomeric proanthocyanidin, epigallocatechin gallate, spirulina, curcumin, and Antrodia camphorata is about 12˜30:1:4:2:1:1.

2. The composition as claimed in claim 1, wherein the ratio of fermented soy extract, oligomeric proanthocyanidin, epigallocatechin gallate, spirulina, curcumin, and Antrodia camphorata is about 12:1:4:2:1:1.

3. The composition as claimed in claim 1, wherein the fermented soy extract is made with the fermentation of an aqueous soy extract with at least one lactic bacteria and/or at least one yeast.

4. The composition as claimed in claim 3, wherein the lactic bacteria are Lactobacillus species.

5. The composition as claimed in claim 1, wherein the oligomeric proanthocyanidin is a grape seed proanthocyanidin extract.

6. The composition as claimed in claim 1, wherein the epigallocatechin gallate is a green tea epigallocatechin gallate (EGCG).

7. (canceled)

8. (canceled)

9. The composition as claimed in claim 1, further comprising a pharmaceutically acceptable carrier, or excipient.

10. (canceled)
